(19) United States
(12) Patent Application Publication
(10) Pub. No.: US 2025/0113795 A1
(43) Pub. Date: Apr. 10, 2025

(54) IMPROVED SOYBEAN EXPLANT PREPARATION AND TRANSFORMATION

(71) Applicant: INARI AGRICULTURE TECHNOLOGY, INC., Cambridge, MA (US)

(72) Inventors: TEJINDER KUMAR MALL, WEST LAFAYETTE, IN (US); LARISSA ARRAIS GUIMARAES, WEST LAFAYETTE, IN (US); CHRISTOPHER ALEXANDER BAGLEY, WEST LAFAYETTE, IN (US)

(21) Appl. No.: 18/730,113

(22) PCT Filed: Jan. 19, 2023
(86) PCT No.: PCT/US2023/060873

§ 371 Date: Jul. 18, 2024

Related U.S. Application Data
(60) Provisional application No. 63/266,963, filed on Jan. 20, 2022.

(51) Int. Cl. A01H 4/00; C12N 15/82

(52) U.S. Cl. CPC A01H 4/005; C12N 15/8201

(57) ABSTRACT

The disclosure relates to methods of preparing soybean explants. During explant preparation, mature seeds are typically first imbibed in water. Instead of using dry seeds directly for imbibition, the moisture content of the seeds is first partially increased. The provided methods significantly diminish imbibition-caused soybean seed cracking and improve explant viability.

### CROSS-REFERENCE TO RELATED APPLICATIONS

This International Patent application claims the benefit of U.S. Provisional Patent Application No. 63/266,963, filed Jan. 20, 2022, which is incorporated herein by reference in its entirety.

### BACKGROUND

Recent advances in genome editing technologies have provided opportunities for precise modification of the genome in many types of organisms, including plants and animals. For example, technologies based on genome editing proteins, such as zinc finger nucleases, TALENs, and CRISPR systems are advancing rapidly and it is now possible to target genetic changes to specific DNA sequences in the genome. Methods of preparing explants for transformation are especially useful for genome editing as well as genetic engineering technologies.

### SUMMARY

Methods of preparing a soybean explant, comprising: (i) pre-hydrating a plurality of dry soybean seeds to obtain partially hydrated soybean seeds wherein moisture content is increased relative to the dry soybean seeds; (ii) soaking the partially hydrated soybean seeds from (i) in water or an aqueous solution to obtain fully hydrated soybean seeds; and (iii) isolating explants from the fully hydrated soybean seeds of (ii) are provided. In certain embodiments, the methods further comprise introducing a polynucleotide into at least one cell of the explant.

### BRIEF DESCRIPTION OF THE DRAWINGS

FIG. 1A-B illustrates cracked seeds used in quantification. Examples of cracks through the seed coat are circled. FIG. 1A shows seeds from a treated sample and FIG. 1B shows the control.

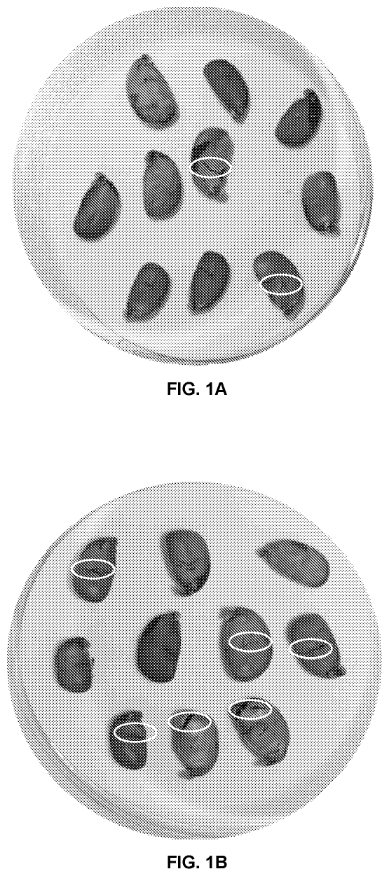

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, flowers, or stalks. In contrast, some plant cells are not capable of being regenerated to produce plants and are referred to herein as “non-regenerable” plant cells.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

During soybean transformation explant preparation, mature seeds are typically first imbibed in water for about 16 to 24 hours. During this process, some seeds show fissures and cracks in the cotyledon tissue especially around the hypocotyl area. The fissures and cracks when large enough can result in the transformation explant breaking and becoming unusable for transformation.

Improved methods of preparing a soybean explant are provided herein. Instead of using dry seeds directly for imbibition, the moisture content of the seeds is first increased. The methods include the steps of: (i) pre-hydrating a plurality of dry soybean seeds to obtain partially hydrated soybean seeds wherein moisture content is increased relative to the dry soy bean seeds; (ii) soaking the partially hydrated soybean seeds from (i) in water or an aqueous solution to obtain fully hydrated soybean seeds; and (iii) isolating explants from the fully hydrated soybean seeds of (ii). The provided methods significantly diminish imbibition-caused soybean seed cracking, particularly for seed lots of a low starting moisture content. In certain embodiments, the recovery of fully hydrated soybean seeds lacking fissures in the seed coat is increased relative to a control method wherein soybean seeds are fully hydrated without a pre-hydration step as in step (i). In certain embodiments, the viability of the explants is increased relative to a control method wherein soybean seeds are fully hydrated without a pre-hydration step as in step (i).

A step of the methods include pre-hydrating a plurality of dry soybean seeds to obtain partially hydrated soybean seeds wherein moisture content is increased relative to the dry soybean seeds.

In certain embodiments, the dry soybean seeds are pre-hydrated by incubating the dry soybean seeds on a semi-solid media comprising a support matrix and water. A non-limiting example of a suitable pre-soaking imbibition semi-solid media formulation is provided in the working Examples. In certain embodiments, the semi-solid media comprises agar, water, a buffer, one or more salts, one or more vitamins, and a carbon source. In embodiments, the semi-solid media comprises Murashige and Skoog basal medium and about 15 grams per liter to about 25 grams per liter sucrose. In certain embodiments, the dry soybean seeds are incubated with the semi-solid medium for at least about 6 hours, typically for about 6 hours to about 8 hours.

In certain embodiments, the dry soybean seeds are pre-hydrated by incubating the dry soybean seeds in a humidity chamber. In certain embodiments, the humidity chamber has a relative humidity of at least about 60%, at least about 70%, at least about 80%, at least about 90%, at least about 95%, or about 100%. In embodiments, the humidity chamber has a relative humidity of about 70% to about 100%. In certain embodiments, the dry soybean seeds are incubated in a humidity chamber for at least about 6 hours, typically for about 6 hours to about 8 hours.

In certain embodiments, the dry soybean seeds are pre-hydrated by incubating the dry soy bean seeds on the semi-solid media in the humidity chamber. In certain embodiments, the dry soybean seeds are incubated with the semi-solid medium in the humidity chamber for at least about 6 hours, typically for about 6 hours to about 8 hours.

The moisture content of the dry soybean seeds may be less than about 10%, less than about 9%, less than about 8%, less than about 7%, or less than about 6% by weight. In embodiments, the moisture content of the dry soybean seeds is about 3% to about 10% by weight. In embodiments, the moisture content of the dry soybean seeds is about 3%, 4%, or 5% to about 6%, 7%, 8%, 9%, or 10% by weight.

The expected moisture content of the partially hydrated soybean seeds varies depending on the beginning moisture content of the dry soybean seeds. The pre-hydrating may increase the moisture content of the seeds to at least about 10%, at least about 11%, at least about 12%, at least about 13%, at least about 14%, or at least about 15% by weight. In certain embodiments, the pre-hydrating increases the moisture content of the seeds to about 7%, about 8%, about 9%, about 10%, or about 11% to about 12%, about 13%, about 14%, about 15%, about 16%, about 18%, or about 20% by weight.

The pre-hydrating may increase the moisture content of the seeds by at least about 2%, at least about 3%, at least about 4%, or at least about 5% relative to the dry soybean seeds by weight. In certain embodiments, the pre-hydrating increases the moisture content of the seeds by about 1% to about 10% relative to the dry soybean seeds, by about 2% to about 6% relative to the dry soybean seeds, or by about 3% to about 5% relative to the dry soybean seeds, all by weight.

A step of the method includes soaking the partially hydrated soybean seeds in water or an aqueous solution to obtain fully hydrated soybean seeds. The soaking may be in water or in an aqueous solution comprising water and one or more solutes, such as a salt, a vitamin, or a buffer. In embodiments, the soaking is for about 16 hours to about 24 hours. In certain embodiments, the fully hydrated soybean seeds comprise a moisture content of about 50% to about 70% by weight. In embodiments, the fully hydrated soybean seeds comprise a moisture content of about 50%, about 55%, or about 60% to about 65% or about 70% by weight.

Moisture content of dry seeds, partially hydrated seeds, and fully hydrated seeds may be measured using any method known in the art such as, for example, oven drying, Karl Fischer titration, or by use of a moisture meter. Basic methods for determining moisture content in seeds are those in which a weighed seed sample is heated in an oven at a specified temperature for a specified time or until the sample attains constant weight. The weight loss that occurs during drying, calculated on a percentage basis, is taken to be the percentage of moisture in the seeds before drying.

Embodiments of the method further include the step of introducing a polynucleotide into at least one cell of the explant. In certain embodiments, gene editing or other molecules (e.g., comprising a polynucleotide, polypeptide or combination thereof) are introduced into at least one cell of the explant.

Gene editing molecules of use in the methods provided herein include molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA or donor or other DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a nCas9 nickase, a type V Cas nuclease, a Cas12a nuclease, a nCas12a nickase, a Cas12d (CasY), a Cas12e (CasX), a Cas12b (C2cl), a Cas12c (C2c3), a Cas12i, a Cas12j, a Cas12L, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) donor DNA template polynucleotides suitable for insertion at a break in genomic DNA by homology-directed repair (HDR) or microhomology-mediated end joining (MMEJ); and (e) other DNA templates (e.g., dsDNA, ssDNA, or combinations thereof) suitable for insertion at a break in genomic DNA (e.g., by non-homologous end joining (NHEJ).

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). In certain embodiments, an RNA-guided endonuclease that leaves a blunt end following cleavage of the target site is used. Blunt-end cutting RNA-guided endonucleases include Cas9, Cas12c, Cas12i, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e. All of the patent publications referenced in this paragraph are incorporated herein by reference in their entirety.

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, plants or plant cells used in the systems, methods, and compositions provided herein can comprise a transgene that expresses a CRISPR endonuclease (e.g., a Cas9, a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas 12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites.

In certain embodiments, zinc finger nucleases or zinc finger nickases can also be used in the methods provided herein. Zinc-finger nucleases are site-specific endonucleases comprising two protein domains: a DNA-binding domain, comprising a plurality of individual zinc finger repeats that each recognize between 9 and 18 base pairs, and a DNA-cleavage domain that comprises a nuclease domain (typically Fok1). The cleavage domain dimerizes in order to cleave DNA; therefore, a pair of ZFNs are required to target non-palindromic target polynucleotides. In certain embodiments, zinc finger nuclease and zinc finger nickase design methods which have been described (Urnov et al. (2010) Nature Rev. Genet., 11:636-646; Mohanta et al. (2017) Genes vol. 8,12: 399; Ramirez et al. Nucleic Acids Res. (2012); 40 (12): 5560-5568; Liu et al. (2013) Nature Communications, 4:2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally-occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fok1. This endonuclease must dimerize to cleave DNA. Thus, cleavage by Fok1 as part of a ZFN requires two adjacent and independent binding events, which must occur in both the correct orientation and with appropriate spacing to permit dimer formation. The requirement for two DNA binding events enables more specific targeting of long and potentially unique recognition sites. Fok1 variants with enhanced activities have been described and can be adapted for use in the methods described herein; see, e.g., Guo et al. (2010) J. Mol. Biol., 400:96-107.

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fok1, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8,12:399). TALE nickases have also been described and can be adapted for use in methods described herein (Wu et al.; Biochem Biophys Res Commun. (2014); 446 (1): 261-6; Luo et al; Scientific Reports 6, Article number: 20657 (2016)).

Various treatments can be used for delivery of gene editing molecules and/or other molecules to a plant cell. In certain embodiments, one or more treatments is employed to deliver the gene editing or other molecules (e.g., comprising a polynucleotide, polypeptide or combination thereof) into a eukaryotic or plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell is soaked in a liquid genome editing molecule-containing composition, whereby the agent is delivered to the plant cell. In certain embodiments, the agent-containing composition is delivered using negative or positive pressure, for example, using vacuum infiltration or application of hydrodynamic or fluid pressure. In certain embodiments, the agent-containing composition is introduced into a plant cell, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the agent-containing composition to a plant cell include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the agent-containing composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633). Any of these techniques or a combination thereof are alternatively employed on the plant explant, plant part or tissue or intact plant (or seed) from which a plant cell is optionally subsequently obtained or isolated; in certain embodiments, the agent-containing composition is delivered in a separate step after the plant cell has been isolated.

In general, the methods described herein result in regenerated plantlets, e.g., soybean plantlets. In embodiments, the method provides soybean plantlets including cells comprising a modification of the endogenous genomic DNA. In these embodiments, the cells comprising a modification of the endogenous genomic DNA can give rise to further generations of seeds and plants that also contain the modification of the endogenous genomic DNA.

Embodiments of the method further include the step of growing the regenerated plantlet (such as a soybean plant comprising the genomic modification or a transformed soybean plant) to maturity, thus providing a mature transformed (T0) plant (such as a mature transformed TO soybean plant). In embodiments, the mature transformed T0 soybean plant has a genome that contains the genomic modification and that is greater than 99.9% identical to that of the embryo of the source soybean seed. Embodiments of the method further include the step of recovering progeny (T1) soybean seeds from the mature transformed (T0) soybean plant. Additional embodiments of the method further include the step of growing a progeny transformed (T1) soybean plant from the T1 soybean seed. Thus, related embodiments include the mature transformed T0 soybean plants, progeny T1 soybean seeds, and progeny T1 soybean plants, all of which contain the genomic modification or are transformed. In embodiments, the transformed soy bean plantlet or plant is of an elite soybean germplasm, or is of an inbred soybean line.

In embodiments, the method provides a regenerated transformed soybean plant containing at least one genetic modification effected by a transformation agent that is absent in the embryo of the source seed (or of the source plant from which the source seed was obtained); depending on the transformation agents used, the genetic modification can be variously characterized as transient transformation, stable genomic changes, gene editing (genome editing), base editing; single or multiplexed genetic changes. In embodiments of the method, the transformation agent includes an RNA-guided nuclease, and the plant contains a genome that has been edited by the RNA-guided nuclease; specific embodiments include a soybean plant that contains in its genome one or more “genome edits” such as deletion of one or more nucleotides, insertion of one or more nucleotides, insertion of a nucleotide sequence encoded by a donor polynucleotide, allele substitution or replacement, and combinations of such genomic changes.

### EMBODIMENTS

The following numbered embodiments also form part of the present disclosure:
1. A method of preparing a soybean explant, comprising: (i) pre-hydrating a plurality of dry soybean seeds to obtain partially hydrated soybean seeds wherein moisture content is increased relative to the dry soybean seeds; (ii) soaking the partially hydrated soybean seeds from (i) in water or an aqueous solution to obtain fully hydrated soybean seeds; and (iii) isolating explants from the fully hydrated soybean seeds of (ii).
2. The method of embodiment 1, wherein the fully hydrated soybean seeds lack fissures in the seed coat of the soybean seeds.
3. The method of embodiment 1 or embodiment 2, wherein the recovery of fully hydrated soybean seeds lacking fissures in the seed coat is increased relative to a control method wherein soybean seeds are fully hydrated without a pre-hydration step as in step (i).
4. The method of any one of embodiments 1 to 3, wherein the viability of the explants is increased relative to a control method wherein soybean seeds are fully hydrated without a pre-hydration step as in step (i).
5. The method of any one of embodiments 1 to 4, wherein the pre-hydrating comprises incubating the dry soybean seeds on a semi-solid media comprising a support matrix and water, incubating the dry soybean seeds in a humidity chamber, or incubating the dry soybean seeds on the semi-solid media in a humidity chamber.
6. The method of embodiment 5, wherein the semi-solid media comprises agar, water, a buffer, one or more salts, one or more vitamins, and a carbon source.
7. The method of embodiment 5 or embodiment 6, wherein the semi-solid media comprises Murashige and Skoog basal medium and about 15 grams per liter to about 25 grams per liter sucrose.
8. The method of any one of embodiments 5 to 7, wherein the incubating is for about 6 hours to about 8 hours.
9. The method of any one of embodiments 5 to 8, wherein the humidity chamber has a relative humidity of at least about 70%.
10. The method of any one of embodiments 1 to 9, wherein the soaking is in water.
11. The method of any one of embodiments 1 to 9, wherein the soaking is in an aqueous solution comprising water and one or more solutes, optionally wherein the solute(s) comprise a salt, a vitamin, and/or a buffer.
12. The method of any one of embodiments 1 to 11, wherein the soaking is for about 16 hours to about 24 hours.
13. The method of any one of embodiments 1 to 12, wherein the moisture content of the dry soybean seeds is less than about 10% by weight, optionally wherein the moisture content of the dry soybean seeds is less than about 9%, less than about 8%, less than about 7%, or less than about 6% by weight.
14. The method of any one of embodiments 1 to 13, wherein the moisture content of the dry soybean seeds is about 3% to about 10% by weight.
15. The method of any one of embodiments 1 to 14, wherein the pre-hydrating increases the moisture content of the seeds to at least about 10% by weight, optionally wherein the pre-hydrating increases the moisture content of the seeds to at least about 11%, at least about 12%, at least about 13%, at least about 14%, or at least about 15% by weight.
16. The method of any one of embodiments 1 to 15, wherein the pre-hydrating increases the moisture content of the seeds to about 7%, about 8%, about 9%, about 10%, or about 11% to about 12%, about 13%, about 14%, about 15%, about 16%, about 18%, or about 20% by weight.
17. The method of any one of embodiments 1 to 16, wherein the pre-hydrating increases the moisture content of the seeds by at least about 2%, at least about 3%, at least about 4%, or at least about 5% by weight relative to the dry soybean seeds.
18. The method of any one of embodiments 1 to 17, wherein the pre-hydrating increases the moisture content of the seeds by about 2% or about 3% to about 4%, about 5%, or about 6% by weight relative to the dry soybean seeds.
19. The method of any one of embodiments 1 to 18, wherein the fully hydrated soybean seeds comprise a moisture content of about 50%, 55%, or 60% to about 65% or 70% by weight.
20. The method of any one of embodiments 1 to 19, wherein the method further comprises introducing a polynucleotide into at least one cell of the explant.
21. The method of embodiment 20, wherein the polynucleotide comprises a guide RNA, a ribonucleoprotein complex comprising a guide RNA and a RNA-guided nuclease, a polynucleotide encoding a guide RNA and/or a RNA-guided nuclease, a polynucleotide encoding a site specific nuclease selected from the group consisting of an artificial zinc finger nuclease or a transcription activator-like effector nuclease (TALEN) protein, and/or a donor DNA template.
22. The method of embodiment 20 or embodiment 21, wherein the method further comprises isolating a plant cell comprising a modification of the endogenous genomic DNA of the plant cell.
23. The method of any one of embodiments 20 to 22, wherein the method further comprises regenerating callus, a plant part, or a plant comprising the modification.
24. The method of any one of embodiments 20 to 23, further comprising selecting at least one cell of the explant that has been transformed with the polynucleotide or portion thereof to obtain a transformed soybean cell.
25. The method of any one of embodiments 20 to 24, further comprising regenerating a transformed soybean plant from the transformed soybean cell.
26. The method of any one of embodiments 20 to 25, wherein the polynucleotide introduction efficiency, the genomic DNA modification efficiency, and/or the transformation efficiency is increased relative to a control method wherein an explant derived from a soybean seed that has not been pre-hydrated to partially increase its moisture content prior to soaking is used.
27. The method of any one of embodiments 20 to 26, wherein the polynucleotide introduction comprises bacterial-mediated polynucleotide transfer, optionally wherein the introducing comprises Agrobacterium-mediated polynucleotide introduction.
28. The method of any one of embodiments 20 to 27, wherein the polynucleotide introduction comprises biolistic-mediated polynucleotide introduction, viral vector-mediated introduction, and/or transfection.
29. The method of any one of embodiments 20 to 28, wherein the soybean plant is of elite soybean germplasm.
30. The method of any one of embodiments 20 to 29, wherein the soybean plant is an inbred soybean line.
31. The method of any one of embodiments 20 to 30, wherein the transformed soybean plant has a genome that is greater than 99.9% identical to that of the embryo of the source soybean seed.
32. The method of any one of embodiments 20 to 31, further comprising recovering seeds comprising the genomic modification or the transformed soybean plant from the soybean plant comprising the genomic modification or the transformed soybean plant.
33. The method of embodiment 32, further comprising growing a progeny soybean plant from the seed, wherein the progeny comprise the genomic modification or are transformed.

### EXAMPLES

### Example 1

A semi-solid media was developed to allow for a more controlled and slow imbibition process. This controlled imbibition process helps reduce the number of seeds that become unusable from cracking during the imbibition process. Table 1 provides a non-limiting example of a pre-soaking imbibition media formulation that is suited for use in the methods described herein.

TABLE 1
Soybean pre-soaking imbibition media (GM0180).

| Ingredients | Concentration | |
|---|---|---|
| MS Modified Basal Medium with | 2.22 | g/L |
| Gamborg Vitamins | | |
| Sucrose | 20.00 | g/L |
| MES | 0.59 | g/L |
| Noble Agar | 5.00 | g/L |

| | |
|---|---|
| pH | 5.7 |

### Example 2

This example illustrates use of seeds from cold storage with or without pre-imbibition on GM0180 media. The moisture content of seeds before sterilization was 9.5%. The seeds were sterilized in a fume hood using Chlorine gas. Test sterile seeds were placed on solid GM0180 imbibition media plates for about 7 hours. The plates were agitated several times during the incubation so the seeds imbibe evenly. Then, test seeds, as well as control seeds, were submerged in sterile water for 16 hours. Cracking of the seed coat was quantified, which correlates to non-viability of the produced explants. The seed that was imbibed on imbibition media had lower number of cracks compared to seed that was not imbibed on the imbibition media (Table 2).

TABLE 2

| | Total # | # of seed | # of seed | % of seed |
|---|---|---|---|---|
| Treatment | of seeds | with cracks | without cracks | showing cracks |
| Test seeds | 201 | 3 | 198 | 1.5% |
| Control seeds | 204 | 43 | 161 | 21% |

### Example 3

This example illustrates use of seeds stored in humidor. The moisture content of the seeds at the start of the experiment was 9.5%. The seeds were sterilized and placed in a cigar humidor with a tray of water for a week. The relative humidity inside the humidor was about 70%. The moisture content of the seeds after incubation in the humidor became 13%. Test sterile seeds were placed on solid GM0180 imbibition media plates for about 7 hours. The plates were agitated several times during the incubation so the seeds imbibe evenly. Then, test seeds, as well as control seeds, were submerged in sterile water for 16 hours. Incubating the seeds at a high humidity for an extended period eliminated seed cracking due to soaking (Table 3).

TABLE 3

| | Total # | # of seed | # of seed | % of seed |
|---|---|---|---|---|
| Treatment | of seeds | with cracks | without cracks | showing cracks |
| Test seeds | 201 | 0 | 201 | 0% |
| Control seeds | 197 | 0 | 197 | 0% |

### Example 4

This example illustrates use of dry starting seeds. The moisture content of the seeds at the start of the experiment was 9.7%. The seeds were placed in a desiccator for 4 days. The moisture content of the seeds at this point was 7.0%. The seeds were then sterilized. Test sterile seeds were placed on solid GM0180 imbibition media plates for about 7 hours. The plates were agitated several times during the incubation so the seeds imbibe evenly. Then, test seeds, as well as control seeds, were submerged in sterile water for 16 hours. The dry seeds that were imbibed on imbibition media had a lower incidence of cracks compared to seed that was not imbibed on the imbibition media (Table 4).

TABLE 4

| | Total # | # of seed | # of seed | % of seed |
|---|---|---|---|---|
| Treatment | of seeds | with cracks | without cracks | showing cracks |
| Test seeds | 195 | 35 | 160 | 17.9% |
| Control seeds | 201 | 160 | 92 | 79.6% |

The breadth and scope of the present disclosure should not be limited by any of the above-described examples.

What is claimed is:

1. A method of preparing a soybean explant, comprising:
(i) pre-hydrating a plurality of dry soybean seeds to obtain partially hydrated soybean seeds wherein moisture content is increased relative to the dry soybean seeds;
(ii) soaking the partially hydrated soybean seeds from (i) in water or an aqueous solution to obtain fully hydrated soybean seeds; and
(iii) isolating explants from the fully hydrated soybean seeds of (ii).

2. The method of claim 1, wherein the fully hydrated soybean seeds lack fissures in the seed coat of the soybean seeds.

3. The method of claim 1, wherein the recovery of fully hydrated soybean seeds lacking fissures in the seed coat is increased relative to a control method wherein soybean seeds are fully hydrated without a pre-hydration step as in step (i).

4. The method of claim 1, wherein the viability of the explants is increased relative to a control method wherein soybean seeds are fully hydrated without a pre-hydration step as in step (i).

5. The method of claim 1, wherein the pre-hydrating comprises incubating the dry soybean seeds on a semi-solid media comprising a support matrix and water, incubating the dry soybean seeds in a humidity chamber, or incubating the dry soybean seeds on the semi-solid media in a humidity chamber.

6. The method of claim 5, wherein the semi-solid media comprises agar, water, a buffer, one or more salts, one or more vitamins, and a carbon source.

7. The method of claim 5, wherein the semi-solid media comprises Murashige and Skoog basal medium and about 15 grams per liter to about 25 grams per liter sucrose.

8. The method of claim 5, wherein the incubating is for about 6 hours to about 8 hours.

9. The method of claim 5, wherein the humidity chamber has a relative humidity of at least about 70%.

10. The method of claim 1, wherein the soaking is in water.

11. The method of claim 1, wherein the soaking is in an aqueous solution comprising water and one or more solutes, optionally wherein the solute(s) comprise a salt, a vitamin, and/or a buffer.

12. The method of claim 1, wherein the soaking is for about 16 hours to about 24 hours.

13. The method of any one of claims 1 to 12, wherein the moisture content of the dry soybean seeds is less than about 10% by weight, optionally wherein the moisture content of the dry soybean seeds is less than about 9%, less than about 8%, less than about 7%, or less than about 6% by weight.

14. The method of claim 1, wherein the moisture content of the dry soybean seeds is about 3% to about 10% by weight.

15. The method of any one of claims 1 to 12, wherein the pre-hydrating increases the moisture content of the seeds to at least about 10% by weight, optionally wherein the pre-hydrating increases the moisture content of the seeds to at least about 11%, at least about 12%, at least about 13%, at least about 14%, or at least about 15% by weight.

16. The method of any one of claims 1 to 12, wherein the pre-hydrating increases the moisture content of the seeds to about 7%, about 8%, about 9%, about 10%, or about 11% to about 12%, about 13%, about 14%, about 15%, about 16%, about 18%, or about 20% by weight.

17. The method of any one of claims 1 to 12, wherein the pre-hydrating increases the moisture content of the seeds by at least about 2%, at least about 3%, at least about 4%, or at least about 5% relative to the dry soybean seeds by weight.

18. The method of any one of claims 1 to 12, wherein the pre-hydrating increases the moisture content of the seeds by about 2% or about 3% to about 4%, about 5%, or about 6% relative to the dry soybean seeds by weight.

19. The method of any one of claims 1 to 12, wherein the fully hydrated soybean seeds comprise a moisture content of about 50%, 55%, or 60% to about 65% or 70% by weight.

20. The method of claim 1, wherein the method further comprises introducing a polynucleotide into at least one cell of the explant.

21. The method of claim 20, wherein the polynucleotide comprises a guide RNA, a ribonucleoprotein complex comprising a guide RNA and a RNA-guided nuclease, a polynucleotide encoding a guide RNA and/or a RNA-guided nuclease, a polynucleotide encoding a site specific nuclease selected from the group consisting of an artificial zinc finger nuclease or a transcription activator-like effector nuclease (TALEN) protein, and/or a donor DNA template.

22. The method of claim 20, wherein the method further comprises isolating a plant cell comprising a modification of the endogenous genomic DNA of the plant cell.

23. The method of claim 20, wherein the method further comprises regenerating callus, a plant part, or a plant comprising the modification.

24. The method of claim 20, further comprising selecting at least one cell of the explant that has been transformed with the polynucleotide or portion thereof to obtain a transformed soybean cell.

25. The method of claim 20, further comprising regenerating a transformed soybean plant from the transformed soybean cell.

26. The method of any one of claims 20 to 25, wherein the polynucleotide introduction efficiency, the genomic DNA modification efficiency, and/or the transformation efficiency is increased relative to a control method wherein an explant derived from a soybean seed that has not been pre-hydrated to partially increase its moisture content prior to soaking is used.

27. The method of any one of claims 20 to 25, wherein the polynucleotide introduction comprises bacterial-mediated polynucleotide transfer, optionally wherein the introducing comprises Agrobacterium-mediated polynucleotide introduction.

28. The method of any one of claims 20 to 25, wherein the polynucleotide introduction comprises biolistic-mediated polynucleotide introduction, viral vector-mediated introduction, and/or transfection.

29. The method of any one of claims 20 to 25, wherein the soybean plant is of elite soybean germplasm.

30. The method of any one of claims 20 to 25, wherein the soybean plant is an inbred soy bean line.

31. The method of any one of claims 20 to 25, wherein the transformed soybean plant has a genome that is greater than 99.9% identical to that of the embryo of the source soybean seed.

32. The method of any one of claims 20 to 25, further comprising recovering seeds comprising the genomic modification or the transformed soybean plant from the soybean plant comprising the genomic modification or the transformed soybean plant.

33. The method of claim 32, further comprising growing a progeny soybean plant from the seed, wherein the progeny comprise the genomic modification or are transformed.
